(19) United States
(12) Patent Application Publication
(10) Pub. No.: US 2024/0216671 A1
(43) Pub. Date: Jul. 4, 2024

(54) CATHETER BLOOD PUMP SHROUDS AND ASSEMBLY THEREOF

(71) Applicant: Shifamed Holdings, LLC, Campbell, CA (US)

(72) Inventors: Brian D. BRANDT, Morgan Hill, CA (US); Michael CALOMENI, San Jose, CA (US)

(21) Appl. No.: 18/554,756

(22) PCT Filed: Apr. 6, 2022
(86) PCT No.: PCT/US2022/023624

§ 371 Date: Oct. 10, 2023

Related U.S. Application Data
(60) Provisional application No. 63/171,691, filed on Apr. 7, 2021.

(51) Int. Cl. A61M 60/13; A61M 25/00; A61M 60/857

(52) U.S. Cl. CPC A61M 60/13; A61M 25/0009; A61M 25/0023; A61M 60/857; A61M 2025/0024; A61M 2207/10

(57) ABSTRACT

Catheter blood pumps that include an expandable pump portion. The pump portions include a collapsible blood conduit that defines a blood lumen. The collapsible blood conduits include a collapsible scaffold adapted to provide radial support to the blood conduit. The pump portion also includes one or more impellers. The collapsible scaffold and/or an elongate member extending proximally from the pump portion may include portions of differing radial stiffness or flexibility.

### CROSS REFERENCE TO RELATED APPLICATIONS

This application claims the benefit of U.S. Application No. 63/171,691, filed Apr. 7, 2021, titled “CATHETER BLOOD PUMP SHROUDS AND ASSEMBLY THEREOF” which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. There is a need for ways to improve the volume or stability of cardiac outflow for these patients, especially during corrective procedures.

Intra-aortic balloon pumps (IABP) are commonly used to support circulatory function, such as treating heart failure patients. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

An IABP commonly works by being placed within the aorta and being inflated and deflated in counterpulsation fashion with the heart contractions, and one of the functions is to attempt to provide additive support to the circulatory system.

More recently, minimally-invasive rotary blood pumps have been developed that can be inserted into the body in connection with the cardiovascular system, such as pumping arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient.

The smallest rotary blood pumps currently available can be percutaneously inserted into the vasculature of a patient through an access sheath, thereby not requiring surgical intervention, or through a vascular access graft. A description of this type of device is a percutaneously-inserted ventricular support device.

There is a need to provide additional improvements to the field of ventricular support devices and similar blood pumps for treating compromised cardiac blood flow.

### SUMMARY OF THE DISCLOSURE

The disclosure is related to intravascular blood pump and their methods of and manufacture.

A method of manufacturing an expandable and collapsible blood conduit for a catheter blood pump is provided, which can include depositing a coating on inner and outer surfaces of a scaffold in an expanded configuration to create a coated scaffold, the coated scaffold comprising a plurality of coated elongate members that define a plurality of apertures.

In some embodiments, depositing the coating comprises spraying the coating on the inner and outer surfaces of the scaffold. Alternatively, depositing the coating comprises spraying the coating while rotating the scaffold. In some examples, depositing the coating occurs while the scaffold is disposed about a mandrel such that inner surfaces of elongate members of the scaffold are not in contact with the mandrel. Additionally, depositing the coating can comprise depositing a coating that comprises a hydrophilic material. In some embodiments, depositing a coating on inner and outer surfaces of the scaffold comprises dipping the scaffold in a coating material, optionally a polymeric material (e.g., a polyurethane).

The method can further include positioning the coated scaffold about an inner tubular member. In some examples, positioning the coated scaffold about an inner tubular member comprises positioning the coated scaffold about an inner tubular member that is disposed on a mandrel. In another embodiment, positioning the coated scaffold about an inner tubular member comprises positioning the coated scaffold about an inner tubular member that comprises a hydrophilic material. In some examples, positioning further comprises positioning a heat shrink tubular member about the coated scaffold and heating the heat shrink tubing to bond the scaffold coating to the inner tubular member.

In some embodiments, the method can further include adhering the coating on the coated scaffold to the inner tubular member to form a conduit. In some embodiments, adhering the coating on the coated scaffold to the inner tubular member comprises creating a blood conduit.

Optionally as part of the method, subsequent in time to the adhering step, the method can include depositing a hydrophilic material on the conduit. In some embodiments, depositing a hydrophilic material on the conduit reduces a sheathing force needed to collapse the collapsible conduit within a sheath not greater than 14 F (and optionally less than 14 F inner diameter), relative to a sheathing force without the hydrophilic material. In other embodiments, depositing a hydrophilic material on the conduit also deposits the hydrophilic material onto one or more struts (e.g., an exemplary strut 14 is labeled in FIGS. 1, 3, and 4) that extend from the conduit.

In one embodiment, the method can include performing an etching process (optionally comprising plasma etching) on the scaffold earlier in time than the depositing step.

In some examples, the scaffold comprises a plurality of struts, and wherein the depositing step deposits the coating on outer surfaces of the plurality of struts, and optionally also on inner surfaces of the plurality of struts. In one embodiment, ends of the plurality of struts are radially closer to an inner mandrel than a central section of the scaffold during the depositing step.

In some examples, the manufactured blood conduit is adapted to be collapsed and sheathed within a sheath 14 F or less (and optionally less than 14 F inner diameter) with less than 15 pounds of sheathing force.

In various embodiments, the manufactured blood conduit is adapted to be collapsed and sheathed within a sheath 14 F or less (and optionally less than 14 F inner diameter) with less than 10 pounds of sheathing force.

An expandable and collapsible blood conduit for a catheter blood pump is also provided, comprising an inner membrane layer defining an inner surface of the blood conduit, an outer hydrophilic layer about the inner membrane layer, and an expandable and collapsible scaffold disposed between the outer hydrophilic layer and the inner membrane layer.

In some embodiments, the inner membrane layer comprises a different material than the outer hydrophilic layer.

In other embodiments, the inner membrane layer comprises a hydrophilic material.

In one example, the scaffold including a plurality of elongate members with etched outer surfaces.

In other embodiments, the inner membrane layer is thicker than the outer hydrophilic layer.

In some embodiments, the blood conduit is adapted to be collapsed and sheathed within a sheath 14 F or less (and optionally less than 14 F inner diameter) with less than 15 pounds of sheathing force, and optionally less than 10 pounds of sheathing force.

In some examples, one or more struts (e.g., 14) have a hydrophilic material therein, wherein the hydrophilic material may be an extension of the hydrophilic layer.

### BRIEF DESCRIPTION OF THE DRAWINGS

FIG. 1 is a side view of an exemplary collapsible and expandable blood conduit.

FIGS. 2A and 2B illustrate an exemplary step for manufacturing a collapsible and expandable blood conduit that includes positioning a tubular member or tubing over a mandrel.

FIG. 3 illustrates a subsequent manufacturing step of positioning a coated scaffold onto the tubular member/mandrel assembly from FIG. 2B.

FIG. 4 illustrates an optional manufacturing step of providing lamination reinforcement, such as along an interface between scaffold and tubing.

FIG. 5 is a flowchart illustrating a method of manufacturing a blood conduit of a blood pump.

### DETAILED DESCRIPTION

Some intravascular blood pumps may include a pump portion that is collapsible and expandable, examples of which are described in the Appendix herein and in the disclosures of the following PCT publications: WO2018/226991, WO2019/094963, WO2019/152875, WO2020 028537, and WO 2020/073047, the disclosures of which are incorporated by reference herein for all purposes. The collapsible pump portion may include a collapsible blood conduit and one or more collapsible impellers therein. The collapsible and expandable blood conduit may include one or more scaffolds and one or layers of material (e.g., a membrane) that together form the blood conduit through which blood is pumped. The disclosures incorporated by reference herein provide exemplary collapsible and expandable blood conduits, and of which may be manufactured using any methods herein as well as include any features of manufactured blood conduits herein.

The disclosure herein is related to expandable and collapsible shrouds or blood conduits that may be incorporated into a pump portion of catheter blood pumps. The disclosure includes methods of manufacturing the shrouds, the shrouds themselves, pump portions that comprise the shrouds, and their methods of use. Expandable pump portions should be reliably collapsible within an outer sheath for delivery and removal, and methods of manufacture are needed that produce shrouds that safely provide the desired pump performance as well as facilitate reliable sheathing.

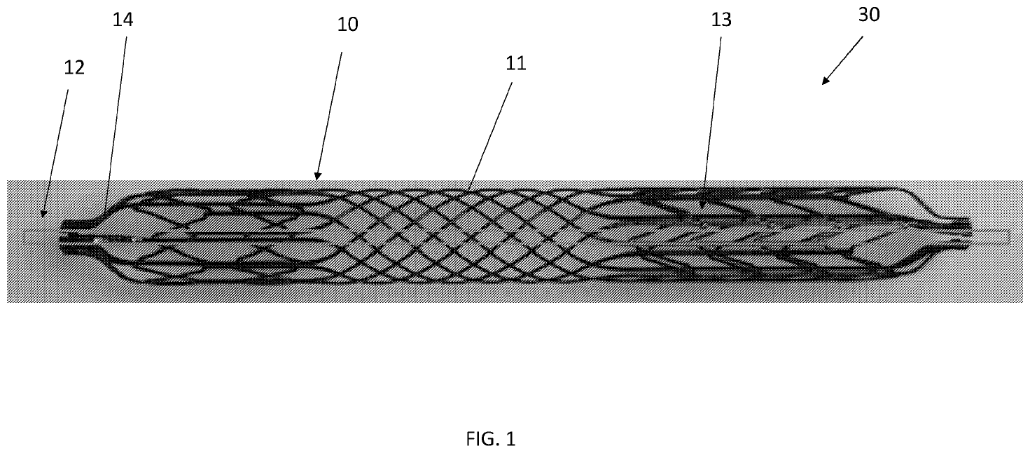

FIGS. 1-4 illustrate exemplary method of manufacturing steps for collapsible and expandable blood conduits or shrouds that may find use within pump portions of catheter blood pumps.

FIG. 1 illustrates a merely exemplary scaffold 10 disposed about an inner mandrel 12, wherein the inner mandrel is not in contact with elongate members 11 of scaffold 10. Scaffold 10 may be, for example, a laser cut member that is cut from a tube, expanded to the expanded configuration shown, and shape set in the expanded configuration. The shape set scaffold may optionally undergo a polishing step, and may be etched, such as with plasma etching. Etching the scaffold may be generally considered to be part of a process that prepares the scaffold for a subsequent depositing or coating step.

The method may include depositing a coating (which may also be referred to herein as coating) on inner and outer surfaces of scaffold 10 to create a coated scaffold 30. The coated scaffold 30 comprises a plurality of coated elongate members 11 that define a plurality of apertures.

FIG. 1 may be considered to show uncoated scaffold 10, but it is also representative of a coated scaffold 30, wherein the coated elongate members 11 define apertures 33. Depositing the coating in the arrangement of FIG. 1 in which mandrel 12 is not in contact with inner surfaces of the elongate members 11 of scaffold 10 allows the coating to be deposited on the inner and outer surfaces of elongate members 11. Mandrel 12 can help stabilize scaffold 10 during the depositing/coating step, and yet does not interfere with coating the inner surfaces of the elongate members 11. This can help ensure reliable and complete coating on the scaffold during the depositing step. This may be considered to be in contrast to an initial step in which a scaffold is disposed snugly about a tubular member initially, followed by a subsequent coating or depositing step, which may be prone to trapping air bubbles between inner surfaces of the scaffold and an inner membrane layer. The depositing step may comprise spraying the coating on the scaffold, and optionally moving the scaffold to help ensure complete coverage of inner and outer surfaces of the scaffold. The coating (and any of the constituent materials therein) may comprises a polymeric material, such as a polyurethane, and may also optionally include one or more hydrophilic materials, which may increase lubricity and ease sheathing, described in more detail herein.

As an alternative to spraying a coating onto the scaffold, depositing a coating on a scaffold may comprise dipping the scaffold into a coating material, which may not require the inner manifold that is shown in FIG. 1. In examples in which the depositing step comprises a dipping step, the scaffold is considered to be a coated scaffold with a plurality of coated elongate members 11 that define a plurality of apertures. It is understood that depositing a coating may comprise multiple different types of steps, such as a spaying step and a dipping step, for example, wherein the depositing yields a coated scaffold with a plurality of coated elongate members 11 that define a plurality of apertures.

The coated scaffold (which includes apertures 33) can then be further processed as set forth below.

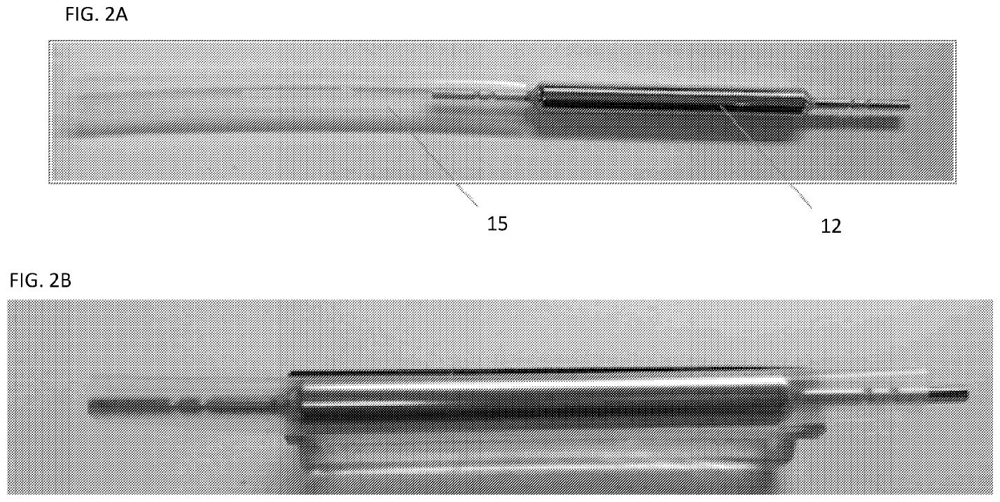

FIGS. 2A and 2B illustrate an exemplary step that includes positioning a tubular member or tubing 15 over a mandrel 12, as shown. The tubular member may form part of the blood conduit, and may define an inner surface thereof thorough which blood is pumped. The tubular member may be created from extrusion or spraying process, for example. The tubular member may comprise any of the polymeric materials herein (e.g., a polyurethane), and may also include a hydrophilic material.

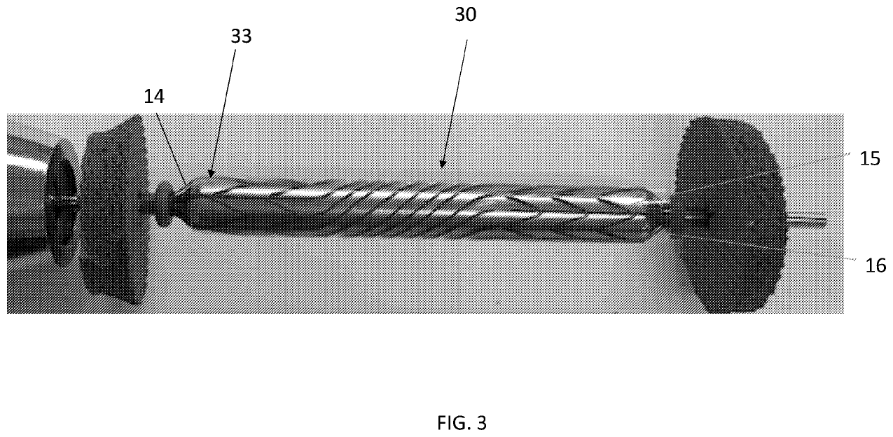

After the tubing 15 is loaded on the mandrel 12 as shown in FIG. 2B, FIG. 3 illustrates a subsequent step of positioning the coated scaffold 30 (described with respect to FIG. 1) onto the tubular member 15 and mandrel 12 from FIG. 2B. A heat-shrink tubing 16 can then be placed over the coated scaffold 30. The assembly shown in FIG. 3 can be exposed to heat, and pressure from the heat-shrink tubing results in the coating on the coated scaffold adhering or bonding to the underlying tubular member (tubing) shown in FIGS. 2A and 2B. The heat-shrink tubing can then be removed after bonding. The bonding or adhering step generally creates a conduit, which may be considered the finished blood conduit, but which may also and likely undergo one or more processing steps to create the finished conduit.

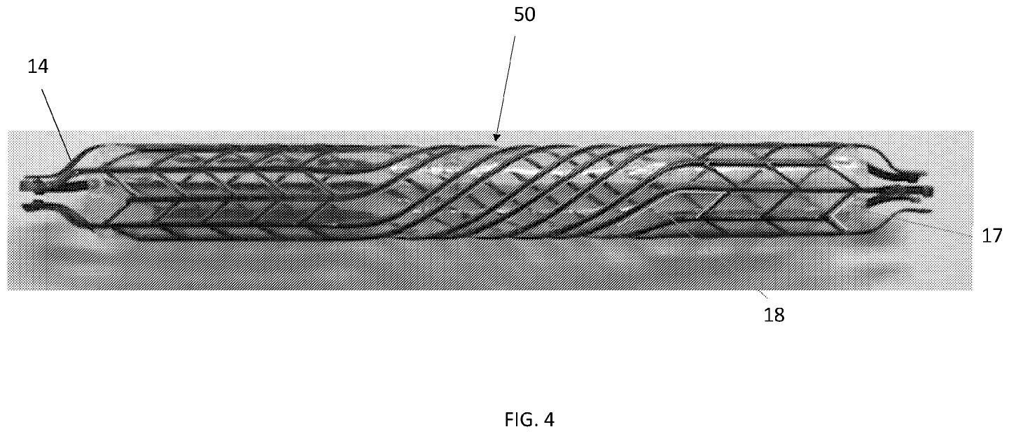

The use of the term conduit in this context thus includes the possibility of one or more processing steps. For example only, the material that creates the conduit may be trimmed 17 at one or both ends, an exemplary location of which is shown in FIG. 4. Optional lamination reinforcement 18 may also take place, such as along interface of scaffold and tubing, an exemplary location of which is shown in FIG. 4.

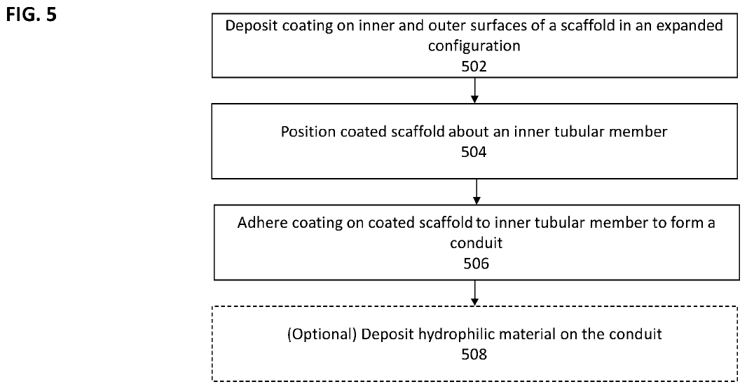

FIG. 5 illustrates a flowchart describing a method of manufacturing an expandable and collapsible blood conduit for a catheter blood pump. Referring to step 502 of the flowchart, the method can comprise depositing a coating on inner and outer surfaces of a scaffold in an expanded configuration to create a coated scaffold, the coated scaffold comprising a plurality of coated elongate members that define a plurality of apertures.

In some embodiments, depositing the coating comprises spraying the coating on the inner and outer surfaces of the scaffold. Alternatively, depositing the coating comprises spraying the coating while rotating the scaffold. In some examples, depositing the coating occurs while the scaffold is disposed about a mandrel such that inner surfaces of elongate members of the scaffold are not in contact with the mandrel. Additionally, depositing the coating can comprise depositing a coating that comprises a hydrophilic material. In some embodiments, depositing a coating on inner and outer surfaces of the scaffold comprises dipping the scaffold in a coating material, optionally a polymeric material (e.g., a polyurethane).

At step 504, the method can further include positioning the coated scaffold about an inner tubular member. In some examples, positioning the coated scaffold about an inner tubular member comprises positioning the coated scaffold about an inner tubular member that is disposed on a mandrel. In another embodiment, positioning the coated scaffold about an inner tubular member comprises positioning the coated scaffold about an inner tubular member that comprises a hydrophilic material. In some examples, positioning further comprises positioning a heat shrink tubular member about the coated scaffold and heating the heat shrink tubing to bond the scaffold coating to the inner tubular member.

At step 506, the method can further include adhering the coating on the coated scaffold to the inner tubular member to form a conduit. In some embodiments, adhering the coating on the coated scaffold to the inner tubular member comprises creating a blood conduit.

At optional step 508, subsequent in time to the adhering step, the method can include depositing a hydrophilic material on the conduit. In some embodiments, depositing a hydrophilic material on the conduit reduces a sheathing force needed to collapse the collapsible conduit within a sheath not greater than 14 F (and optionally less than 14 F inner diameter), relative to a sheathing force without the hydrophilic material. In other embodiments, depositing a hydrophilic material on the conduit also deposits the hydrophilic material onto one or more struts (e.g., an exemplary strut 14 is labeled in FIGS. 1, 3, and 4) that extend from the conduit.

In one embodiment, the method can include performing an etching process (optionally comprising plasma etching) on the scaffold earlier in time than the depositing step.

In some examples, the scaffold comprises a plurality of struts, and wherein the depositing step deposits the coating on outer surfaces of the plurality of struts, and optionally also on inner surfaces of the plurality of struts. In one embodiment, ends of the plurality of struts are radially closer to an inner mandrel than a central section of the scaffold during the depositing step.

In some embodiments, the manufactured blood conduit is adapted to be collapsed and sheathed within a sheath 14 F or less (and optionally less than 14 F inner diameter) with less than 15 pounds of sheathing force.

In various embodiments, the manufactured blood conduit is adapted to be collapsed and sheathed within a sheath 14 F or less (and optionally less than 14 F inner diameter) with less than 10 pounds of sheathing force.

An optional additional step may include depositing, such as by spraying and/or dipping, another layer of material onto the assembly subsequent in time to removing the heat shrink tubing. For example, one or more hydrophilic materials (e.g., HydroThane™, HydroMed™) may be deposited onto the assembly, which may enhance lubricity of the blood conduit and which may reduce the sheathing force needed to collapse the blood conduit. The optional depositing step may comprise depositing a hydrophilic material onto one or more sets of struts that extend axially from the blood conduit, which may occur at the same the hydrophilic material is deposited onto the conduit. Sheathing tests performed on blood conduits manufactured using methods herein that included a depositing (coating) step followed by lamination with an inner tubular member (e.g., as shown in FIG. 3) resulted in sheathing forces less than 15 pounds, including less than 10 pounds, when collapsed into a sheath less than 14 F in size. Sheathing tests performed on blood conduits manufactured using methods herein that included depositing a hydrophilic material, such as depositing a hydrophilic material after bonding coating on a coated scaffold to an inner tubular material, resulted in sheathing forces less than 10 pounds when collapsed into a sheath less than 14 F in size. In these tests, the sheathing forces were achieved when sheathing within a 14 F inner diameter (“ID”), including within a 12 F ID (including about 10 F ID).

1. A method of manufacturing an expandable and collapsible blood conduit for a catheter blood pump, comprising:
depositing a coating on inner and outer surfaces of a scaffold in an expanded configuration to create a coated scaffold, the coated scaffold comprising a plurality of coated elongate members that define a plurality of apertures;
positioning the coated scaffold about an inner tubular member; and
adhering the coating on the coated scaffold to the inner tubular member to form a conduit.

2. The method of claim 1 wherein depositing the coating comprises spraying the coating on the inner and outer surfaces of the scaffold.

3. The method of claim 1, wherein depositing the coating comprises spraying the coating while rotating the scaffold.

4. The method of claim 1, wherein depositing the coating occurs while the scaffold is disposed about a mandrel such that inner surfaces of elongate members of the scaffold are not in contact with the mandrel.

5. The method of claim 1, where depositing the coating comprises depositing a coating that comprises a hydrophilic material.

6. The method of claim 1, wherein positioning the coated scaffold about an inner tubular member comprises positioning the coated scaffold about an inner tubular member that is disposed on a mandrel.

7. The method of claim 1, wherein positioning the coated scaffold about an inner tubular member comprises positioning the coated scaffold about an inner tubular member that comprises a hydrophilic material.

8. The method of claim 1, further comprising,
subsequent in time to the adhering step, depositing a hydrophilic material on the conduit.

9. The method of claim 8, wherein depositing a hydrophilic material on the conduit reduces a sheathing force needed to collapse the collapsible conduit within a sheath not greater than 14 F, relative to a sheathing force without the hydrophilic material.

10. The method of claim 8, wherein depositing a hydrophilic material on the conduit also deposits the hydrophilic material onto one or more struts that extend from the conduit.

11. The method of claim 1, further comprises performing an etching process on the scaffold earlier in time than the depositing step.

12. The method of claim 1, wherein positioning further comprises positioning a heat shrink tubular member about the coated scaffold and heating the heat shrink tubing to bond the scaffold coating to the inner tubular member.

13. The method of claim 1, wherein the scaffold comprises a plurality of struts, and wherein the depositing step deposits the coating on outer surfaces of the plurality of struts, and optionally also on inner surfaces of the plurality of struts.

14-18. (canceled)

19. An expandable and collapsible blood conduit for a catheter blood pump, comprising:
an inner membrane layer defining an inner surface of the blood conduit;
an outer hydrophilic layer about the inner membrane layer; and
an expandable and collapsible scaffold disposed between the outer hydrophilic layer and the inner membrane layer.

20. The blood conduit of claim 19, wherein the inner membrane layer comprises a different material than the outer hydrophilic layer.

21. The blood conduit of claim 19, wherein the inner membrane layer comprises a hydrophilic material.

22. The blood conduit of claim 19, wherein the scaffold including a plurality of elongate members with etched outer surfaces.

23. The blood conduit of claim 19, wherein the inner membrane layer is thicker than the outer hydrophilic layer.

24. The blood conduit of claim 19, wherein the blood conduit is adapted to be collapsed and sheathed within a sheath 14 F or less with less than 15 pounds of sheathing force, and optionally less than 10 pounds of sheathing force.

25. The blood conduit of claim 19, wherein one or more struts have a hydrophilic material therein, wherein the hydrophilic material may be an extension of the hydrophilic layer.
